(19) United States
(12) Patent Application Publication
(10) Pub. No.: US 2013/0023829 A1
(43) Pub. Date: Jan. 24, 2013

(54) HYPODERMIC NEEDLE RECAPPER

(71) Applicant: Kristin L. Carrigan, Chicopee, MA (US)

(72) Inventor: Kristin L. Carrigan, Chicopee, MA (US)

(73) Assignee: ANSELL LIMITED, Richmond (AU)

(21) Appl. No.: 13/487,851

(22) Filed: Jun. 4, 2012

Related U.S. Application Data
(60) Provisional application No. 61/510,953, filed on Jul. 22, 2011.

(51) Int. Cl. A61M 5/32

(52) U.S. Cl. 604/192

(57) ABSTRACT

A hypodermic needle recapper, comprising a substantially planar substrate formed of a given material and having a given thickness which, in combination, cause the substrate to be substantially impervious to penetration of a hypodermic needle. The substrate defining an opening having a flexible perimeter adapted to admit a hypodermic needle cap therein and having a plurality of flexible flaps formed about a periphery of the opening so as to provide the flexibility to the perimeter of the opening. Each flap has a thickness that is less than the given thickness of the substrate.

### CROSS-REFERENCE TO RELATED APPLICATIONS

This application claims priority to U.S. Provisional Patent Application Ser. No. 61/510,953, filed Jul. 22, 2011, which is incorporated by reference herein in its entirety.

### BACKGROUND OF THE INVENTION

1. Field of the Invention

Embodiments of the present invention generally relate to a hypodermic needle recapper, and more particularly, to a hand-held apparatus for safely recapping a hypodermic needle.

2. Description of the Related Art

Approximately 385,000 sharps injuries are incurred by hospital employees each year, according to the Centers for Disease Control and Prevention (CDC). Studies show that about 12% of these injuries occurring in surgical settings are caused by unsafe needle devices and improper handling of needles and other sharps syringes. One way to help prevent accidental needle sticks is to provide a removable cap over the syringe needle. The syringe user can easily remove the cap in a safe manner, however, putting the cap back on can be very problematic.

An officially recommended method commonly used today for recapping a hypodermic needle in a manner that provides a safe and inexpensive means to protect users from inadvertent skin puncture, is called the “scoop method”. This method comprises holding the syringe with one hand and sliding the tip of the needle into a needle cap that is lying on a flat surface. This method allows a user to re-cap the needle while only using one hand, thereby greatly decreasing the possibility of an accidental needle stick.

Although the scoop method is generally considered to be a safe way to recap a needle, it is in fact a cumbersome technique and fraught with stability issues. That is, while a user attempts to single-handedly scoop-up the cylindrically shaped cap using the needle, the cap has a tendency to roll and/or slide along the flat surface due to its cylindrically shape, thereby wasting time and possibly disrupting or sticking other equipment on the flat surface. Thus, a handheld apparatus is generally more preferred for needle recapping; however use of handheld apparatus can greatly increase the potential of an inadvertent needle stick.

Presently, a multitude of hypodermic needle recappers are currently being used, either in a hand-held position or placed on a flat surface such as a table or bench.

Various handheld shields and recappers for hypodermic needles are known, however, they all are either too small to provide effective shielding, are somewhat complex in shape or use various different types of materials or techniques in order to effectively grasp the needle cap, thereby undesirably increasing the complexity, dimensions and cost for the recapper.

Therefore, there is a need for a handheld needle recapper that is very simple to manufacture and easy to store, while at the same time is safe, easy to use and offers additional advantages.

### SUMMARY OF THE INVENTION

Embodiments of the present invention provide a hypodermic needle recapper, comprising a substantially planar substrate formed of a material and having a thickness which, in combination, cause the substrate to be substantially impervious to penetration of a hypodermic needle, the substrate defining an opening having a flexible perimeter adapted to admit a hypodermic needle cap therein, and a plurality of flexible flaps formed about a perimeter of the opening so as to provide the flexibility to the perimeter of the opening. Each flap has a thickness that is less than the thickness of the substrate. In some embodiments, the thickness of the substrate is reduced by an equal amount at both a front and back side of the substrate. In other embodiments, the thickness is reduced at only one of the front and back sides.

### BRIEF DESCRIPTION OF THE DRAWINGS

FIG. 1 illustrates a front perspective view of a hypodermic needle recapper according to one or more embodiments, in combination with an optional holder;

FIGS. 2A, 2B, 2C and 2D illustrate perspective views of one way of using the hypodermic needle recapper of FIG. 1;

FIGS. 3A, 3B and 3C illustrate structural details of the recapper of FIG. 1; and

FIG. 4 illustrates a perspective view of an alternative embodiment of the recapper according to FIG. 1.

While the method and apparatus described herein is by way of example for several embodiments and illustrative drawings, those skilled in the art will recognize that the method and apparatus for providing a hypodermic needle recapper is not limited to the embodiments or drawings described. It should be understood, that the drawings and detailed description thereto are not intended to limit embodiments to the particular form disclosed. Rather, the intention is to cover all modifications, equivalents and alternatives falling within the spirit and scope of the method and apparatus for providing a hypodermic needle recapper as defined by the appended claims. Any headings used herein are for organizational purposes only and are not meant to limit the scope of the description or the claims. As used herein, the word “may” is used in a permissive sense (i.e., meaning having the potential to), rather than the mandatory sense (i.e., meaning must). Similarly, the words “include”, “including”, and “includes” mean including, but not limited to.

### DETAILED DESCRIPTION OF EMBODIMENTS

Embodiments of the present invention generally comprise a method and apparatus for preventing needle stick during recapping of a hypodermic needle. In one embodiment, a hypodermic needle recapper comprises a substantially planar substrate formed of a material and having a thickness which, in combination, cause the substrate to be substantially impervious to penetration of a hypodermic needle. The substrate defines an opening having a flexible perimeter adapted to admit a hypodermic needle cap therein, and a plurality of flexible flaps formed about a perimeter of the opening so as to provide the flexibility to the perimeter of the opening. Each flap has a thickness that is less than the thickness of the substrate and has a distal end extending radially in toward a center of the opening. In some embodiments, the thickness of the substrate is reduced by an equal amount at both a front and back side of the substrate, in other embodiments, the thickness is reduced at only one of the front and back sides.

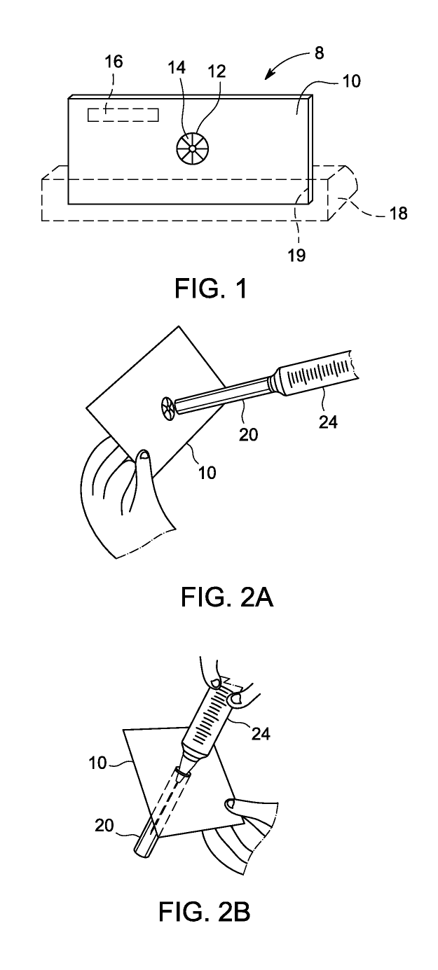

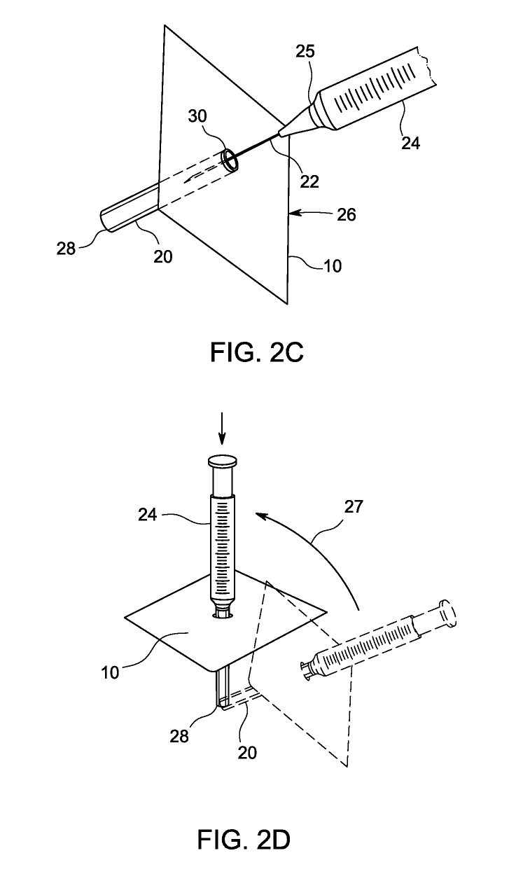

FIG. 1 illustrates a front perspective view of a hypodermic needle recapper according to one or more embodiments, in combination with an optional holder. As shown in the FIG. 1 embodiment, a needle recapper 8 includes a planar substrate 10 that serves as a protective shield, having through opening 12 formed therein, adapted to capture and hold a hypodermic needle cap 20 (shown in FIG. 2). In some embodiments, the opening 12 may be a through hole with surrounding flaps 14.

Printing, such as instructions for use, may be located on a front surface portion 16 of the substrate 10. Optionally, as shown by dashed lines in FIG. 1, a holder 18 may be provided in which the substrate 10 may be placed so as to hold substrate 10 in an upright position. Holder 18 may comprise a planar base adapted to lay flat or be adhesively attached to a support surface, and includes a slot 19 formed therein and having a length, width and depth adapted to receive an edge of substrate 10 therein. When an edge of substrate 10 is received in slot 19, block 18 provides stable vertical support for substrate 10.

FIGS. 2A, 2B, 2C and 2D illustrate perspective views of one way of using the hypodermic needle recapper of FIG. 1. In use, the recapper 8 is placed on a flat surface, in optional holder 18 or is held in the hand of the user at the edges, with the fingers of the user kept a distance away from the opening 12, as shown by FIGS. 2A through 2C. FIGS. 2A and 2B show a user inserting the needle cap 20 through the opening 12 so as to prepare to uncap a hypodermic needle 22 of a syringe 24. Once inserted, the syringe 24 may be placed in a stable “at-ready” temporary position formed by positioning an end portion 28 of cap 20 and an edge 26 portion of substrate 10 on a support surface, leaving the end of syringe 24 that is opposite to the needle 22 tilted upward or downward and ready for use during a surgical procedure, When the syringe 24 is to be used, the fingers of the user may be used to hold the cap 20 on the backside of the recapper while the needle 22 is removed from the cap 20, leaving the cap 20 in the opening 12, as shown in FIG. 2C. If the connection between the cap 20 and the needle 22 is by a press fit, removal is accomplished by the user merely applying forces in opposed longitudinal directions between the cap 20 and the needle 22, respectively. If the connection between cap 20 and the needle 22 comprises engaging corresponding threads, then removal is accomplished by the user merely applying forces in opposed rotational directions to the cap 20 and the needle 22. Depending on the fit and grip between the opening 12 and the needle 20 (or a hub or flange 25 of the syringe that retains the needle 20 therein), holding of the cap 20 by the users fingers may not be necessary. After the needle 22 is fully withdrawn from the cap 20, the recapper 8 and cap 20 can be placed in the stable “at-ready” temporary position noted above, leaving an open end 30 of cap 20 tilted upward and ready to receive needle 22 during recapping, as shown in FIGS. 2C and 2D. Note, a support surface, such as a tray, is not shown in either of FIGS. 2C or 2D.

A reverse of the above uncapping procedure is used to recap the needle 22. As illustrated by the technique shown in FIGS. 2C and 2B, during recapping the user has one hand on the needle and one hand may hold a corner of the substrate 10, as shown in FIG. 2B. Next, the needle 22 is inserted into cap 20, as shown by FIG. 2C, and then re-secured to syringe 24 by application of longitudinal or rotation forces, as required by the fit between the cap 20 and syringe 24. Because the user's hand at an edge of substrate 10, the risk of an accidental needle stick during recapping according to this technique is greatly reduced.

Alternatively, the user may not touch substrate 10 at all and simply insert the needle 22 into the cap 20 while the substrate 10 and cap 20 are temporarily resting on the support surface, as shown by dashed lines in FIG. 2D. After the needle 22 is inserted, and with the cap still resting on the support surface, the syringe 24 is tilted vertically, as shown by the arrow 27. At this point, the user presses on the syringe 24 to cause the cap 20 to snap back onto the syringe 24 and securely cover needle 22. Because the user's hand is only on the syringe and doesn't even touch the cap 20 or substrate 10, the risk of an accidental needle stick during recapping is even further reduced.

The substrate 10 may be provided in different sizes, for example with the size of a business card, i.e., 2 inches tall by 3 inches wide or larger, such as 3 to 4 inches tall by 4 to 6 inches long, with a thickness of about 1/16 of an inch, although other dimensions could be used to adapt substrate 10 to other uses and environments.

The substrate 10 may be made of a thermoplastic polymer, so as to be tough, yet flexible, resistant to fatigue and can withstand a large range of temperatures. Thus, the material used to form substrate 10 may be polypropylene (e.g., PP recyclable Number 5) or polycarbonate.

The result is a recapper of low cost construction, perfect as a disposable/single patient use product. By use of the opening 12 with flaps, the recapper 8 is able to receive the cap of any standard size syringe.

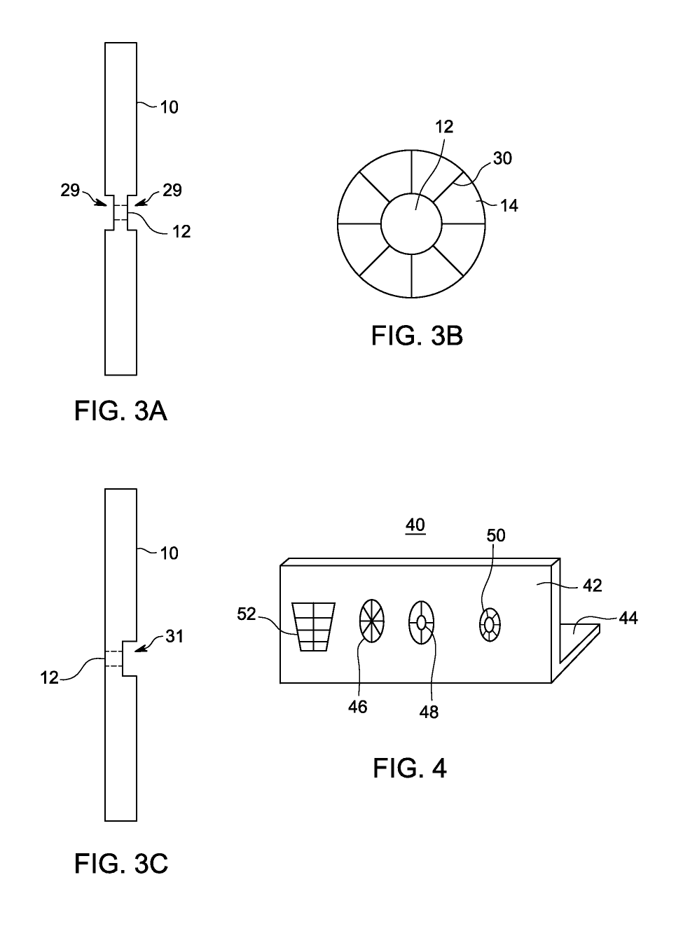

FIGS. 3A, 3B and 3C illustrate structural details of the recapper of FIG. 1. FIGS. 3A through 3C show details of recapper 8. FIG. 3A shows a side view of recapper 8 where substrate 10 is thinned by a substantially equal amount on its opposite sides in a circular area 29 centered around opening 12. After thinning, as shown in FIG. 3B, slots 30 are formed in the thinned material equidistantly around opening 12, thereby forming the flexible flaps 14. FIG. 3C shows an alternative embodiment, where only one side of substrate 10 is thinned in a circular area 31 centered around opening 12, for forming the flexible flaps 14.

FIG. 4 illustrates a perspective view of an alternative embodiment of the recapper according to FIG. 1. FIG. 4 shows a further embodiment for a recapper in accordance with the present invention, wherein the substrate 10 of recapper 40 has an L shape, comprising a front wall 42 and a bottom 44. This L shape allows the recapper 40 to stand upright without use of a holder 18 as shown in FIG. 1. An underside portion of bottom 44 could include an adhesive material so that the recapper 40 can be temporarily affixed to a support surface, not specifically shown. Multiple openings of different shapes and sizes, with or without flaps, may be used for retaining a needle cap 20. The flaps, if used, may be full flaps that extend entirely to the center of the opening, as shown by opening 46, or the flaps may be shortened into flap segments similar to flaps 14 shown in FIG. 3B, leaving a small diameter center through hole, as shown by openings 48 and 50. Additionally, one or more of the openings may be substantially “slot-like”, such as shown by the tapered slot 52. Tapered slot 52 also includes a plurality of flaps attached about the perimeter of opening 52 so that their free ends opposed each other at the mid-point of the slot 52. When removing a cap from a syringe needle using a tapered slot 52, the cap 20 or needle hub 25 may be placed through the opening near the wider top-end portion of the slot 52, and then moved to the narrower, lower-end of the slot 52, until the cap 20 becomes firmly wedged and held in place, thereby allowing the cap to be removed from the hub by the user either unscrewing or withdrawing the syringe from the cap 20. It is noted that although a 90 degree L shape is shown in FIG. 4, other angles greater or less than 90 degrees could be used.

Thus, the hypodermic needle recapper shown and described herein is effective to reduce accidental needlestick hazards by providing a safe way for users to handle and recap syringe needles. The present embodiments provide not only a shield to protect the users' hands from potential needlesticks, but also a safer area to place syringes when not in use, as well as a way to easily recap a syringe needle using only one hand. Furthermore, the described embodiments comply with requirements set by the Occupational Safety and Health Administration (OSHA) to use engineering controls (i.e. a mechanical device) for recapping of hypodermic needles.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. For example, as noted above, the opening 12 may be a through hole, with surrounding flaps 14 or without surrounding flaps 14 or similar holding elements. The planar substrate 10 may be formed of a material or materials other plastic, such as fiberglass or even sheet metal. Furthermore, a metallic flat washer or similar device (not specifically shown) may be placed on the front or back surface of the substrate 10 surrounding the opening 12, to assist grasping of the cap 20 and/or to provide better puncture resistance to the substrate 10 in the area adjacent to the flaps 14.

Thus the illustrated embodiments were chosen and described in order to best explain the principles of the present disclosure and its practical applications, to thereby enable others skilled in the art to best utilize the invention and various embodiments with various modifications as may be suited to the particular use contemplated. Further embodiments of the invention may be devised without departing from the basic scope of disclosure herein, and the scope thereof is to be determined by the claims that follow.

1. A hypodermic needle recapper, comprising:
a substantially planar substrate formed of a material and having a thickness which, in combination, cause the substrate to be substantially impervious to penetration of a hypodermic needle;
the substrate defining an opening having a flexible perimeter adapted to admit a hypodermic needle cap therein; and
a plurality of flexible flaps formed about the perimeter of the opening so as to provide the flexibility to the perimeter of the opening, wherein each flap has a thickness that is less than the thickness of the substrate.

2. The recapper of claim 1, wherein said substrate comprises a thermoplastic polymer.

3. The recapper of claim 2, wherein said substrate comprises polypropylene.

4. The recapper of claim 1, wherein the opening is a circular aperture and each flap has a distal end extending radially toward a center of the aperture.

5. The recapper of claim 1, wherein said substrate comprises substantially an L shape.

6. The recapper of claim 1, wherein said substrate is thinned by an equal amount on opposite sides thereof to reduce the given thickness of the substrate.

7. The recapper of claim 1, wherein said substrate is thinned on only one side thereof to reduce the thickness of the substrate.

8. The recapper of claim 1, wherein a plurality of openings are defined in the substrate, each opening having a flexible perimeter adapted to admit a hypodermic needle cap therein.

9. The recapper of claim 8, wherein at least two of the plurality of openings have a different flexible perimeter adapted to admit a hypodermic needle cap of a different size therein.

10. A hypodermic needle recapper, comprising:
a substrate formed of a material and having a thickness which, in combination, cause the substrate to be substantially impervious to penetration of a hypodermic needle;
the substrate having an opening therethrough defining a hypodermic needle cap receiving area therein; and
a plurality of flexible flaps formed in the hypodermic needle cap receiving area,
wherein each flap has a thickness that is less than the thickness of the substrate.

11. The recapper of claim 10, wherein said substrate comprises a thermoplastic polymer.

12. The recapper of claim 11, wherein said substrate comprises polypropylene.

13. The recapper of claim 10, wherein the opening in the cap receiving area comprises an aperture, and each flap has a distal end extending radially toward a center of the aperture.

14. The recapper of claim 10, wherein said substrate comprises substantially an L shape.

15. The recapper of claim 10, wherein said substrate is thinned in the cap receiving area by an equal amount on opposite sides thereof to reduce the thickness of the substrate.

16. The recapper of claim 10, wherein said substrate is thinned in the cap receiving area on only one side thereof to reduce the thickness of the substrate.

17. The recapper of claim 1, wherein the substrate has a plurality of openings therein, each opening having a flexible perimeter adapted to admit a hypodermic needle cap therein.

18. The recapper of claim 17, wherein at least two of the plurality of openings have a different flexible perimeter adapted to admit a hypodermic needle cap of a different size therein.

19. The recapper of claim 10, wherein the opening in the cap receiving area comprises a circular aperture, and each flap has a distal end extending radially toward a center of the aperture.

20. The recapper of claim 10, wherein the opening in the cap receiving area comprises a variable width slot, and each flap has a distal end extending toward a center of the slot.
